(19) United States
(12) Patent Application Publication
(10) Pub. No.: US 2020/0147268 A1
(43) Pub. Date: May 14, 2020

(54) PROSTHETIC TISSUE VALVE AND METHOD OF PREPARING THE SAME

(71) Applicant: Peijia Medical (Suzhou) Co., Ltd., Suzhou (CN)

(72) Inventors: Kongrong Karl PAN, Suzhou (CN); Yi ZHANG, Suzhou (CN); Guozheng CHENG, Suzhou (CN); Kun ZHANG, Suzhou (CN); Jinjin ZHANG, Suzhou (CN)

(73) Assignee: Peijia Medical (Suzhou) Co., Ltd., Suzhou (CN)

(21) Appl. No.: 16/525,543

(22) Filed: Jul. 29, 2019

(30) Foreign Application Priority Data
Nov. 8, 2018 (CN) 201811327535.4

(51) Int. Cl. A61L 27/36; A61L 27/38; A01N 1/02

(52) U.S. Cl. CPC A61L 27/3691; A61L 27/3826; A61L 2430/20; A01N 1/0284; A61L 27/3882

(57) ABSTRACT

The present disclosure provides a prosthetic tissue valve and a preparation method thereof. The preparation method consists of a lyophilization process of soaked biological tissues under preset condition to obtain a lyophilized prosthetic tissue valve, which provides a technical support for pre-loading the lyophilized prosthetic tissue valve onto the delivery device immediately after manufacture. The preset conditions may include a cooling process with a cooling rate of which the temperature decreases from room temperature to a lyophilization temperature, the lyophilization temperature of −200° C.-0° C., and a pressure of 1 Pa-102 kPa. In such a way of preparation, the present disclosure can provide a lyophilized prosthetic tissue valve.

### CROSS-REFERENCE TO RELATED APPLICATIONS

The present application claims foreign priority of Chinese Patent Application No. 201811327535.4, filed on Nov. 8, 2018 in the National Intellectual Property Administration of China, the entire contents of which are hereby incorporated by reference.

### TECHNICAL FIELD

The described embodiments relate to the field of techniques of medical devices, and in particular to a preparation method of prosthetic tissue valves.

### BACKGROUND

Dysfunction of heart valves endangers human health and lives, and significantly impacts quality of patients' daily work and life. Conventional therapies include conservative treatments with medicines, as well as surgical replacement of heart valves. Although surgeries significantly improve the prognosis, elder patients are usually suffering from multiple complicated diseases, such as cardiopulmonary dysfunction, and therefore, may not be tolerant to surgeries. Compared with surgeries, involvement of tissue valve prostheses in treatments is minimally invasive, leaves a short period time for recovery, and does not generate scars, which is beneficial to many patients.

Globally, the prosthetic tissue valves must be stored in a certain concentration of glutaraldehyde during the process of preparation, transportation, and utility, which increases the cost for preparation and transportation. In addition, during transportation, the prosthetic tissue valves are separated from the delivery device of the tissue valves. Prosthetic tissue valves shall be loaded onto the delivery device prior to a surgery, which requires rinsing of the tissue valves, and rigorously trained engineers to load onto the delivery device by crimping. The delivery device with tissue valves shall be transferred to a surgical team to introduce into a patient's heart. The process of tissue valve rinsing and loading prior to a surgery is complicated and usually takes approximately half an hour to be finished, which significantly increases the operation time, and may potentially disrupt the sterile conditions of the device and therefore increase possibilities of surgical infection.

### SUMMARY OF THE DISCLOSURE

The present disclosure is to solve the above-mentioned technical problem by providing a method to prepare a prosthetic tissue valve, wherein the prosthetic tissue valve is a lyophilized tissue valve.

To solve the above-mentioned technical problem, the present disclosure is to provide a technical solution, which is a preparation method for a prosthetic tissue valve, including following operations: under preset conditions, performing cooling and lyophilization to soaked tissues to obtain a lyophilized prosthetic tissue valve, which can be a technical support for pre-loading the lyophilized prosthetic tissue valve into the delivery device immediately after manufacture. The preset conditions may include cooling rate and lyophilization temperatures, wherein the lyophilization temperature may be −200° C.-0° C.

Differentiating from current available techniques in the art, the preparation method for prosthetic tissue valves provided in the present disclosure includes: lyophilization of biological tissues after soaking under preset conditions, to obtain “dry” prosthetic tissue valves, wherein the preset conditions may include cooling rate and a lyophilization temperature of −200° C.-0° C. Beneficial effects of the preparation method for prosthetic tissue valves provided in the present disclosure include conservation of the spatial structures of the biological tissues, wherein the prosthetic tissue valves can maintain the softness and bioactivities in the lyophilized form. Preparation of prosthetic tissue valves in such a way may reduce the cost of manufacture and transportation of the prosthetic tissue valves, simplify the operations involved in the utilization, and may provide technical support for pre-loading the lyophilized prosthetic tissue valves into the delivery devices immediately after manufacture

### BRIEF DESCRIPTION OF THE DRAWINGS

In order to clarify embodiments in the present disclosure, appended figures, which are referred in the embodiments, are described in detail as the following. The appended figures described in the following only reflect a part of the embodiments. Without any creative endeavor, skilled personnel in the art may obtain other figures based on the figures included in the present disclosure.

FIG. 1 is an illustrative flow chart of a preparation method for prosthetic tissue valves in one embodiment.

FIG. 2a is a scanning electronic microscopic graph of lyophilized bovine pericardiums in Embodiment 1.

FIG. 2b is a scanning electronic microscopic graph of lyophilized porcine pericardiums in Embodiment 2.

FIG. 2c is a scanning electronic microscopic graph of lyophilized aortic valves in Embodiment 3.

FIG. 2d is a scanning electronic microscopic graph of lyophilized bovine pericardiums in Embodiment 4.

FIG. 3 is an illustrative figure comparing stretching intensity of a “dry” sample with that of a “wet” sample, wherein the samples are provided in 4 of the embodiments.

FIG. 4a shows the appearance of the “wet” sample in Embodiment 1 after 200×10⁶ times of cycles.

FIG. 4b shows the appearance of the “dry” sample in Embodiment 1 after 200×10⁶ times of cycles.

FIG. 5a shows the appearance of the “wet” sample in Embodiment 2 after 200×10⁶ times of cycles.

FIG. 5b shows the appearance of the “dry” sample in Embodiment 2 after 200×10⁶ times of cycles.

FIG. 6a shows the appearance of the “wet” sample in Embodiment 3 after 200×10⁶ times of cycles.

FIG. 6b shows the appearance of the “dry” sample in Embodiment 3 after 200×10⁶ times of cycles.

FIG. 7a shows the appearance of the “wet” sample in Embodiment 4 after 200×10⁶ times of cycles.

FIG. 7b shows the appearance of the “dry” sample in Embodiment 4 after 200×10⁶ times of cycles.

### DETAILED DESCRIPTION

Referring to the appended figures, a precise and complete description of the embodiments is provided in the present disclosure as the following. Apparently, the present disclosure can be implemented by, but not limited to the provided embodiments.

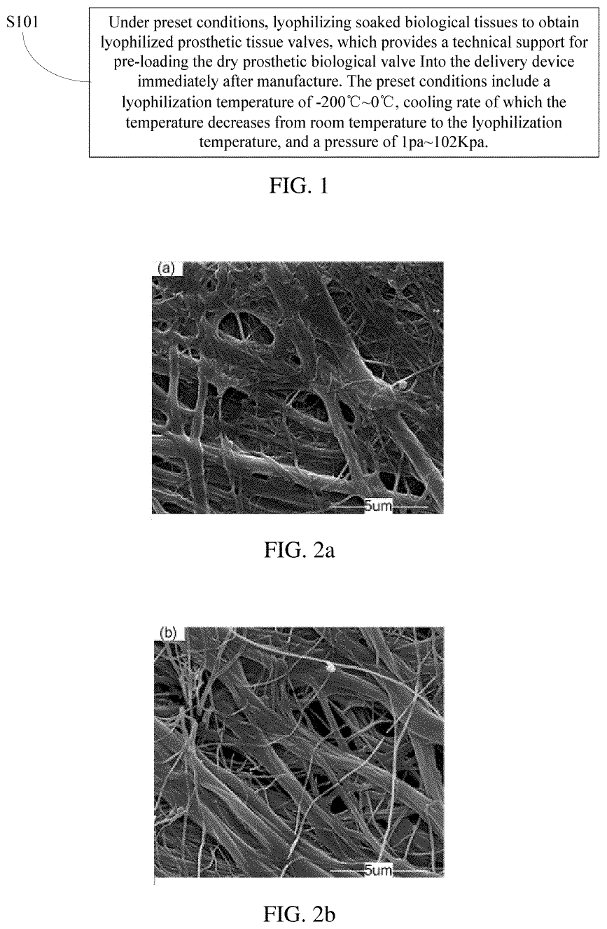

FIG. 1 illustrates a preparation method for prosthetic tissue valves in an embodiment. The method can include operations at the block illustrated in FIG. 1.

A block S101 includes, under preset conditions, cooling and lyophilizing biological tissues after soaking, to obtain lyophilized prosthetic tissue valves, which provides a technical support for pre-loading the lyophilized prosthetic tissue valve into the delivery device immediately after manufacture. The preset conditions may include cooling rate and a lyophilization temperature at −200° C.-0° C.

More specifically, in one embodiment, the biological tissues in S101 may be mammal tissues, which may be anyone from pericardium (such as bovine pericardium, porcine pericardium, equine pericardium, pericardium from donkeys, and the like), aortic valves, mitral valves, tricuspid valves, pulmonary valves, skin, and tissues from venous valved conduits. In other embodiments, the biological tissues may be from other sources, and should not be limited by the present disclosure.

In another embodiment, the above-mentioned cooling rate may be 0.5° C./min-30° C./s, and the lyophilization temperature may be at −200° C.-0° C., such as 0° C., −5° C., −10° C., −15° C., −20° C., −25° C., −30° C., −35° C., −40° C., −50° C., −60° C., −70° C., −80° C., −90° C., −100° C., −150° C., −200° C., and the like.

In another embodiment, the above-mentioned present conditions include pressure and/or time for lyophilization, wherein the pressure may be 1 Pa—102 Kpa, such as 1 Pa, 10 Pa, 20 Pa, 30 Pa, 40 Pa, 50 Pa, 100 Pa, 200 Pa, 500 Pa, 1 KPa, 10 KPa, 50 KPa, 100 KPa, 102 KPa, and the like; and the time may be 4 h—72 h, such as 4 h, 6 h, 8 h, 10 h, 12 h, 14 h, 16 h, 18 h, 20 h, 22 h, 24 h, 30 h, 36 h, 42 h, 48 h, 60 h, 72 h, and the like.

The above mentioned lyophilization process can maximize the conservation of the spatial structure of the biological tissues, and maintain the softness and bioactivity of the biological tissues in the lyophilized form, which further reduce the cost of the preparation and transportation of the prosthetic tissue valves, and simplify the operations involved in the utilization, providing technical support for pre-loading the lyophilized prosthetic tissue valve into the delivery device immediately after manufacture.

Due to individual differences among patients, the size of the appropriate prosthetic tissue valves may be varied. In one embodiment, the lyophilized prosthetic tissue valve may be obtained by the block S101, followed by a cutting process to reach a certain size.

Also, in other embodiments, the process of cutting the prosthetic tissue valve may be performed prior to the block S101. Detailed operations may include soaking biological tissues, followed by cutting the biological tissue into a certain size to obtain a “wet” tissue valve. The block S101 includes lyophilization of the “wet” prosthetic tissue valve under preset conditions to obtain a lyophilized prosthetic tissue valve.

Furthermore, the present disclosure is to provide a prosthetic tissue valve, which may be obtained by a method described in any of the following embodiments. The obtained tissue valves may be applied as aortic valves, mitral valves, tricuspid valves, and pulmonary valves.

Embodiments are provided in the present disclosure to further demonstrate the prosthetic tissue valves and the preparation method thereof. The physical properties of the “wet” and “dry” biological valves provided in the following embodiments are detected under the same evaluation criteria, and the same conditions.

### Embodiment 1

Bovine pericardiums (wet samples) may be provided, and cooled to reach 0° C. at 0.5° C./min, and then lyophilized for 6 hours at the temperature of 0° C. under a pressure of 100 Pa, to obtain a “dry” bovine pericardiums (dry samples). The “dry” bovine pericardiums may be sewn to obtain a heart valve.

### Embodiment 2

Porcine pericardiums (wet samples) may be provided, cooled to reach −5° C. at 0.5° C./min, followed by cooling to reach −20° C. at 5° C./min, and then lyophilized for 12 hours at the temperature of −20° C., under a pressure of 200 Pa, to obtain a “dry” porcine pericardiums (dry samples). The “dry” porcine pericardiums may be sewn to obtain a heart valve.

### Embodiment 3

Aortic valves (wet samples) may be provided, cooled to reach −5° C. at 1° C./min, followed by cooling to reach −40° C. at 30° C./min, and then lyophilized for 48 hours at the temperature of −40° C., under a pressure of 300 Pa, to obtain “dry” aortic valves (dry samples). The “dry” aortic valves may be sewn to obtain a heart valve.

### Embodiment 4

Bovine pericardiums (wet samples) may be provided, cooled to reach −5° C. at 1° C./min, followed by cooling to reach −35° C. at 10° C./s, then cooled to reach −60° C. at 5° C./min, and then lyophilized for 48 hours at the temperature of −60° C. under a pressure of 500 Pa, to obtain “dry” bovine pericardiums (dry samples). The “dry” bovine pericardiums may be sewn to obtain a heart valve.

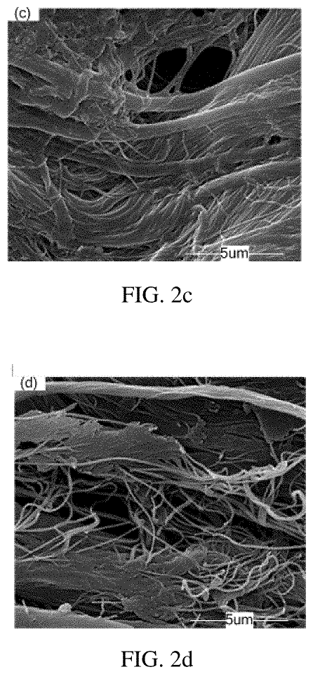

Micro-structures of the lyophilized prosthetic tissue valves obtained from the above embodiments may be observed under scanning electronic microscope, and the microscopic photographs are illustrated in FIG. 2, wherein FIG. 2a is a scanning electronic microscopic graph of the lyophilized bovine pericardiums in Embodiment 1, FIG. 2b is a scanning electronic microscopic graph of the lyophilized porcine pericardiums in Embodiment 2, FIG. 2c is a scanning electronic microscopic graph of the lyophilized aortic valves in Embodiment 3, and FIG. 2d is a scanning electronic microscopic graph of the lyophilized bovine pericardiums in Embodiment 4. These microscopic photographs show that the spatial structures of the lyophilized prosthetic tissue valves obtained from the above four embodiments are completely conserved, the structural integrity of the collagen fibers are conserved, disruption is not shown, and porous structures can be observed.

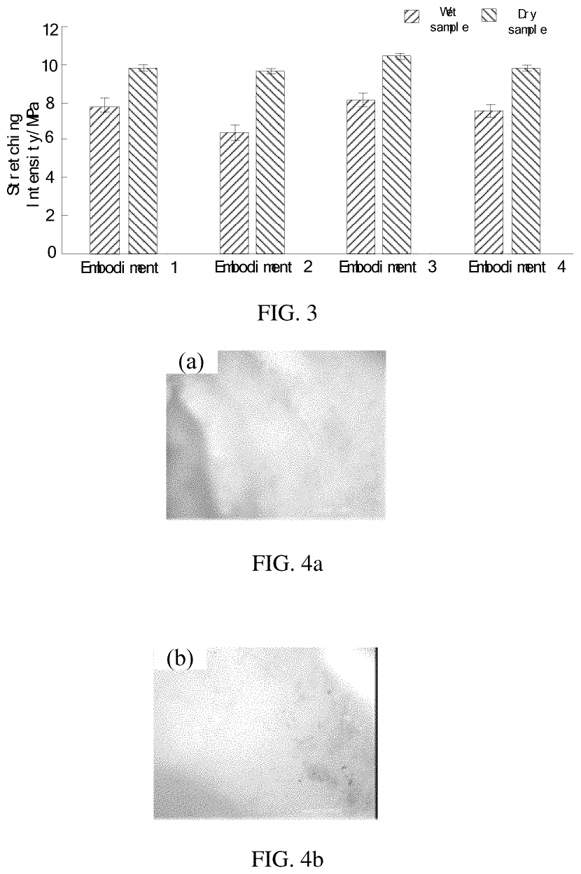

Referring to FIG. 3, the stretching intensities of the “dry” samples obtained from the above embodiments are compared with those of the respective “wet” samples. The “dry” samples obtained from the above four embodiments exhibit significantly higher stretching intensities than the respective wet samples, indicating the lyophilized samples may behave better mechanical properties.

In order to guarantee qualities of prosthetic tissue valves, prior to any clinical use, durability tests should be performed as per GB/T1449.3-2016 standards, including tests for the appearance of the valve leaflets and fluid dynamic tests.

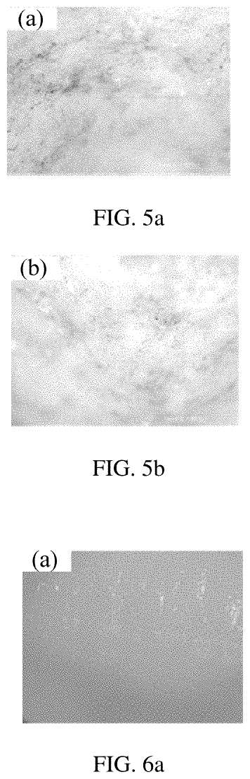

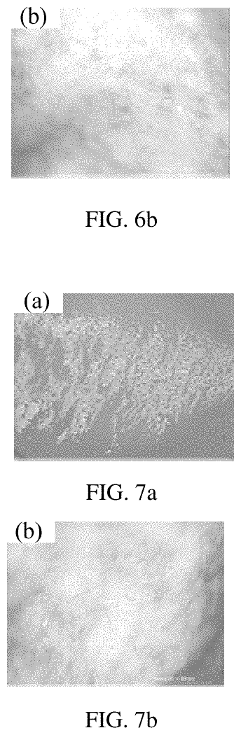

To be specific, FIGS. 4a and 4b show the appearance of the “wet” sample and the “dry” sample obtained from embodiment 1 after 200×10⁶ times of cycles, respectively. FIG. 5a and FIG. 5b show the appearance of the “wet” sample and the “dry” sample obtained from embodiment 2 after 200×10⁶ times of cycles, respectively. FIG. 6a and FIG. 6b show the appearance of the “wet” sample and the “dry” sample obtained from embodiment 3 after 200×10⁶ times of cycles, respectively. FIG. 7a and FIG. 7b show the appearance of the “wet” sample and the “dry” sample obtained from embodiment 4 after 200×10⁶ times of cycles, respectively. Referring to these figures, the lyophilized prosthetic biological valves obtained from the above four embodiments do not show perforation and incomplete fitting, and are not torn, enlarged, sliced, and worn.

The results of the fluid dynamic tests of the “wet” samples and “dry” samples obtained from the four embodiments after 200×10⁶ times of cycles are shown in the following table. The table suggests that effective opening areas (EOA) and total valvular regurgitation of the “dry” samples are comparable to the respective wet samples.

TABLE 1
Durability test results of the samples obtained from the 4 embodiments

| | Embodiment 1 | | Embodiment 2 | | Embodiment 3 | | Embodiment 4 | |
|---|---|---|---|---|---|---|---|---|
| | Wet | Dry | Wet | Dry | Wet | Dry | Wet | Dry |
| Testing Items | samples | samples | samples | samples | samples | samples | samples | samples |
| Effective | 2.59 | 2.59 | 2.51 | 2.54 | 2.46 | 2.50 | 2.39 | 2.44 |
| opening area | | | | | | | | |
| (EOA, cm²) [symbol] | | | | | | | | |
| regurgitation | 9.40 | 8.43 | 9.90 | 6.79 | 6.81 | 5.82 | 5.44 | 5.07 |
| fraction | | | | | | | | |
| (forward | | | | | | | | |
| flow %) [symbol] | | | | | | | | |

To summarize, the properties of the lyophilized prosthetic tissue valves provided in the present disclosure are comparable to those of the respective wet tissue valves, which provides a technical support for pre-loading the lyophilized prosthetic tissue valve into the delivery device immediately after manufacture.

Differentiating from current available techniques in the art, the preparation method for prosthetic tissue valves provided in the present disclosure includes lyophilization of biological tissues after soaking under preset conditions, to obtain lyophilized prosthetic tissue valves. The preparation method for prosthetic tissue valves provided in the present disclosure optimizes the conservation of the spatial structures of the biological tissues, and therefore, the softness and bioactivities of the prosthetic tissue valves can be maintained in the lyophilized form. Preparation of prosthetic tissue valves in such a way may reduce the cost of manufacture and transportation of the prosthetic tissue valves, simplify the operations involved in the utilization, and may provide technical support for pre-loading the lyophilized prosthetic tissue valves into the delivery devices immediately after manufacture.

The present disclosure is to provide, but not limited to the embodiments. The present disclosure includes any structural and process equivalent transformation based on the specification and the appended figures, and any direct and indirect use of the described techniques.

What is claimed is:

1. A method of preparing a prosthetic tissue valve, consisting of, under preset conditions, performing lyophilization to soaked biological tissues, to obtain lyophilized prosthetic tissue valves, which provides a technical support for pre-loading the lyophilized prosthetic tissue valve into the delivery device immediately after manufacture, wherein the preset conditions consist of:
a cooling process with a cooling rate of which the temperature decreases from room temperature to a lyophilization temperature;
the lyophilization temperature of −200° C.-0° C.; and
a pressure of 1 Pa-102 kPa, wherein, after the cooling process, the lyophilization temperature condition and the pressure condition are applied to the biological tissues during the lyophilization at the same time.

2. The method according to claim 1, wherein
the cooling rate is 0.5° C./min-30° C./s; and
the cooling rate is a constant rate during the entire cooling process or variable for different temperature ranges to control crystallization exotherms and sizes and distribution of final crystals.

3. The method according to claim 1, wherein
the lyophilization process lasts for 4 hours-72 hours.

4. The method according to claim 1, wherein the biological tissues are mammalian tissues, wherein the mammalian tissues comprise at least one of the following tissues: mammalian pericardiums, aortic valves, mitral valves, tricuspid valves, pulmonary valves, skins, pleura, and venous valved conduits.

5. The method according to claim 1, wherein the soaked biological tissues are lyophilized under the preset conditions, followed by a cutting process to reach a certain size, to obtain the lyophilized prosthetic tissue valves.

6. The method according to claim 1, wherein
under the preset conditions, prior to the lyophilization, the preparation method further comprises cutting the soaked biological tissues to reach a certain size to obtain wet prosthetic tissue valves; and
under the preset conditions, lyophilization is performed to the biological tissues to obtain the lyophilized tissue valves.

7. A prosthetic tissue valve, which is a lyophilized tissue valve obtained from soaked biological tissues under preset conditions, wherein the preset conditions consist of:
a cooling process with a cooling rate of which the temperature decreases from room temperature to a lyophilization temperature;
the lyophilization temperature of −200° C.-0° C.; and
a pressure of 1 Pa-102 kPa, wherein, after the cooling process, the biological tissues are treated at the lyophilization temperature and the pressure at the same time.

8. The prosthetic tissue valve according to claim 7, wherein
the cooling rate is 0.5° C./min-30° C./s; and
the cooling rate is a constant rate during the entire cooling process or variable for different temperature ranges to control crystallization exotherms and sizes and distribution of final crystals.

9. The prosthetic tissue valve according to claim 7, wherein the lyophilization process lasts for 4 hours-72 hours.

10. The prosthetic tissue valve according to claim 7, wherein the prosthetic tissue valves are used as aortic valves, mitral valves, tricuspid valves, and pulmonary valves.
